(19) United States
(12) Patent Application Publication
(10) Pub. No.: US 2006/0141007 A1
(43) Pub. Date: Jun. 29, 2006

(54) Agent with a retarded release of substances

(76) Inventor: Gunther Beisel, Monheim (DE)

Correspondence Address: FRIEDRICH KUEFFNER, 317 MADISON AVENUE, SUITE 910, NEW YORK, NY, 10017, US

(21) Appl. No.: 10/539,528

(22) PCT Filed: Dec. 9, 2003
(86) PCT No.: PCT/EP03/13924

§ 371 Date: Feb. 13, 2006

(30) Foreign Application Priority Data
Dec. 19, 2002 (DE) 102 59 509.7
Dec. 19, 2002 (DE) 202 19 658.5

(51) Int. Cl. A61K 31/737; A61K 31/728; A61K 47/00

(52) U.S. Cl. 424/439; 514/54

(57) ABSTRACT

The present invention relates to a composition for producing a satiation effect and for weight reduction comprising at least one compound which is swellable and comprises nutrients.

The present invention relates to a composition having delayed release, in particular of nutrients and/or active compounds, which is suitable for producing weight reduction, in particular for use in diet programmes, for example for formula diets, and/or moreover for use in medicinal delayed release compositions.

Numerous experiments have been undertaken, by using medicaments, to break down superfluous accumulations of fat in the human body or to prevent their formation. There are, for example, “appetite suppressants”, which attempt biochemically to suggest to the body an aversion to food intake. These compositions in some cases have appreciable harmful side effects.

In addition to the numerous known diet proposals, there are also mechanical and electromechanical agents, by which a specific breakdown of fat or building up of muscle should take place. The action of such agents, however, is very doubtful.

DE 4025912 discloses a composition for oral consumption, which consists of a container dissolvable in the stomach and releasing the contents. This is filled with a substance which, after its release in the stomach, increases its volume and thereby suggests a feeling of satiation to the body. The disadvantage of this satiating agent is the that the danger of intestinal destruction exists.

Furthermore, DE 199 42 417 discloses spongy preparations containing stably crosslinked compounds, which increase their volume in the stomach and thus produce a feeling of satiation. However, the production of these preparations necessitates additional process steps for the introduction of stable crosslinkages.

On account of continuously increasing health consciousness, however, a further improvement in compositions for producing the weight reduction, in particular for use in diet programmes (e.g. formula diets) is of high medical and economic relevance.

It is an object of the present invention to make available an improved composition for oral consumption which has a higher gastric residence time than known compositions of its type and thereby leads to a more effective weight reduction. Furthermore, it should be suitable for weight reduction with simultaneous regulation of the nutrient uptake and/or release of active compounds, since weight reductions can as a rule be accompanied with a loss of nutritive value and health risks which can make medicinal treatment necessary. Moreover, simple production from inexpensive raw materials which involve no health risks is desirable.

The present object is achieved by a composition comprising at least one compound which is swellable, and comprise nutrients or active compounds or mixtures of nutrients and active compounds which are released in the stomach with a delay.

The use of anionic polymers as compounds which must be swellable according to the invention is preferred. These preferably include polysaccharides, in particular polyuronic acid-containing polysaccharides, and those having a low degree of esterification. Alginic acids, their derivatives and salts (alginates) are particularly preferred. However, it is also possible according to the invention to use all other uronic acid-containing compounds. According to the invention, the use of cellulose or cellulose derivatives is furthermore preferred. The use of synthetic or semisynthetic cellulose derivatives, for example, carboxymethylcellulose or of polyacrylates is conceivable.

Cellulose is to be understood as meaning water-insoluble polysaccharides of the empirical composition (C₆H₁₀O₅)ₙ. To put it more precisely, it is an isotactic β-1,4-polyacetal of cellobiose (4-O-β-D-glucopyranosyl-D-glucose).

Generally, celluloses chemically modified by polymer analogous reactions are defined as cellulose derivatives. They comprise both products in which hydroxyl hydrogen atoms of the anhydroglucose units of the cellulose are substituted exclusively, for example by means of esterification and/or the etherification reactions, by organic or inorganic groups, and those which are formed with formal replacement of hydroxyl groups of the natural polymers by functional groups which are not bonded via an oxygen atom (e.g. deoxycelluloses) or by means of intramolecular elimination of water (anhydrocelluloses, celluloses) or oxidation reactions (aldehyde-, keto- and carboxycelluloses) gebildet werden. Products which are obtained on cleavage of the C₂, C₃ carbon bond of the anhydroglucose units (dialdehyde- and dicarboxycelluloses), in which the monomer unit characteristic of the cellulose is thus no longer intact, are also numbered among the cellulose derivatives. Cellulose derivatives are also accessible by means of other reactions, e.g. by means of crosslinking or graft copolymerization reactions.

According to the invention the use of cellulose or cellulose derivatives as a mixture with pectins is advantageous. Likewise, mixtures comprising alginic acid or its derivatives and pectins are preferred.

Alginic acid is a linear polyurethane acid consisting of variable proportions of D-mannuronic acid and L-guluronic acid, which are linked to one another by β-glycosidic bonds, the carboxyl groups not being esterified. A molecule of alginic acid can be composed of approximately 150-1050 uronic acid units, it being possible for the average molecular weight to vary in a range from 30-200 kDa.

The polysaccharide alginic acid ist a constituent of the cell walls of brown algae. The proportion of alginic acid in the dry matter of the algae can here constitute up to 40%. The alginic acid is obtained by alkaline extraction using methods known per se according to the prior art. The resultant pulverulent alginic acid is thus purely vegetable and has high biocompatibility. It can absorb 300 times the amount of its own weight of water with formation of highly viscous solutions. In the presence of polyvalent cations, alginic acid forms ‘gels’. The formation of alginate gels in the presence of divalent cations, such as calcium or barium, is described in Shapiro I., et al. (Biomaterials, 1997, 18: 583-90). The latter, on account of its toxicity, is not suitable, however, for use in biomedicine.

In addition to calcium chloride, calcium gluconate also yields suitable divalent cations. The use of magnesium salts or a mixture of various physiologically acceptable divalent cations is also conceivable.

Polysaccharides having a low degree of esterification preferably used are pectins, xanthan, tragacanth and chondroitin sulfate having a low degree of esterification.

With respect to the polymers having a low degree of esterification, the use of pectins having a low degree of esterification is particularly preferred according to the invention. Pectins consist of chains of α-1,4-glycosidically linked galacturonic acid units, whose acid groups are 20-80% esterified with methanol. A differentiation is made between highly esterified pectins (>50%) and pectins having a low degree of esterification (<50%). The molar mass varies between 10-500 kDa. Pectins are obtained by acid extraction using methods known per se according to the prior art from the internal constituents of citrus fruit peel, fruit pressing residue or sugar beet pulp. The resulting pectins (apple pectin, citrus pectin) are thus purely vegetable and have high biocompatibility. They can form gels with absorption of water.

The use of pectin gels in the presence of divalent cations, such as calcium or barium, is known. Here too, the latter, on account of its toxicity, however, is not suitable for use in biomedicine. In addition to calcium chloride, calcium gluconate also yields suitable divalent cations. The use of magnesium salts or a mixture of various physiologically acceptable divalent cations is also conceivable.

The use according to the invention of pectins is advantageously distinguished in that these have hypocholesterolemic properties. This property is advantageous within the meaning of the present invention, since overweight is generally accompanied by an increased cholesterol level.

The composition according to the invention furthermore comprises essential nutrients and also vitamins and trace elements. Possible nutrients are in particular vitamins, amino acids, minerals and trace elements.

The composition according to the invention is thus particularly suitable for diets guaranteeing complete nutrition (formula diets). The composition according to the invention on the one hand contains all nutrients and on the other hand a satiating effect is achieved by means of the swellable compounds, in particular in the case of a distended stomach. “Active compounds” are to be understood as meaning, for example, vitamins, trace elements or pharmaceutical active compounds. The following substances are listed by way of example, which, however, are non-limiting for the present invention:

Pharmaceutical active compounds within the meaning of the invention is to be understood as meaning all substances having a pharmaceutical or biological action. Below, examples of active compounds-containing formulations according to the invention from different therapeutic classes are presented, which, however, are non-limiting for the present invention.

Examples of ACE inhibitors are: benazepril, captopril, cilazapril, enalapril, fosinopril, lisinopril, perinodopril, quinapril, ramipril, trandolopril.

Examples of analeptics are: almitrine, amiphenazole, caffeine, doxapram, etamivan, fominoben, metamfetamine, nicethamide, pentetrazole.

Examples of analgesics (opioids) are: alfentanil, buprenorphine, cetobemidone, dextromoramide, dextropropoxyphene, fentanyl, flupirtin, hydromorphone, levomethadone, levorphanol, meptazinol, morphine, nalbuphine, oxycodone, pentazocine, pethidine, piritramide, tilidine, tramadol.

Examples of analgesics (nonopioids) are: acetylsalicylic acid, benzyl mandelate, bucetin, ethenzamide, ketorolac, metamizole, morazone, paracetamol, phenacetin, phenazone, propyphenazone, salicylamide.

Examples of anthelmintics are: albendazole, diethylcarbamazine, mebendazole, praziquantel, tiabendazole. Examples of antiallergics/antihistaminics are: anatazoline, astemnizole, azelastine, bamipine, brompheniramine, buclizine, carbinoxamine, cetirizine, chlorphenamine, clemastine, cyclizine, cyproheptadine, dimenhydramine, doxylamine, fexofenadine ketotifen, loratadine, mepyramine, mizolastine, nedrocromil, oxatomide, oxomemazine, pheniramine, phenyltoloxamine, spaglumic acid, terfenadine, triprolidine.

Examples of antiarrhythmics are: ajmaline, amiodarone, aprindine, quinidine, disopyramide, mexiletine, procainamide, propafenone, tocainide.

Examples of antibiotics/chemotherapeutics are: amikacin, gentamicin, kanamycin, paromomycin, sisomicin, streptomycin, tobramycin, chloroquine, halofantrin, hydroxychloroquine, mefloquine, proguanil, ethambutol, isoniazid, rifabutin, rifampicin, cefacetrile, cefaclor, cefadroxil, cefalexin, cefalotin, cefamandole, cefazolin, cefixime, cefmenoxime, cefoperazone, cefotaxime, cefotetan, cefotiam, cefoxitin, cefpodoxime (proxetil), cefradine, cefsulodin, ceftazidime, ceftizoxime, ceftriaxone, cefuroxime (axetil), latamoxef, cinoxacin, ciprofloxacin, enoxacin, nalidixic acid, norfloxacin, ofloxacin, pipemidic acid, rosoxacin, clarithromycin, erythromycin, roxithromycin, amoxicillin, ampicillin, apalcillin, azidocillin, aziocillin, bacampicillin, benzylpenicillin, carbenicillin, carindacillin, dicloxacillin, flucloxacillin, meziocillin, oxacillin, phenoxymethylpenicillin, piperacillin, pivampicillin, propicillin, ticarcillin, colistin, teicoplanin, vancomycin, cotrimoxazole, sulfamethoxydiazine, doxycycline, oxytetracyline, tetracycline, atovaquone, chloramphenicol, fosfomycin, imipenem, metronidazole, nitrofurantoin, pentamidine, taurolidine, trimethoprim.

Examples of antidepressants are: amitripytyline, amitriptyline oxide, clomipramine, desipramine, dibenzepin, dosulepin, doxepin, fluoxetine, fluvoxamine, imipramine, lithium salts, maprotiline, nomifensine, opipramol, oxitriptan, tranylcypromine, trimipramine, tryptophan.

Examples of antidiabetics/anti-hypoglycemics are: acarbose, carbutamide, chlorpropamide, glibenclamide, glibornuride, gliclazide, glimepiride, glipizide, gliquidone, glisoxepid, glymidine, guar, insulin, mefformin, tolazamide, tolbutamide.

Examples of antidiarrheals are: difenoxin, diphenoxylate, loperamide, petin, tannin.

Examples of antidotes are: flumazenil, naloxone, naltrexone.

Examples of antiemetics are: alizapride, betahistine, thiethylperazine.

Examples of anti-epileptics are: barbexaclone, carbamazepine, ethosuximide, lamotrigine, mepacrine, mesuximide, phenobarbital, phenytoin, primidone, sultiam, trimethadion, valproic acid, vigabatrine.

Examples of anti-fibrinolytics are: aminocaproic acid, 4-(aminomethyl)benzoic acid, tranexamic acid.

Examples of antihypertensives are: clonidine, diazoxide, doxazosin, guanethidine, hydralazine, methyldopa, moxonidine, sodium nitroprusside, phentolamine, prazosin, reserpine, tiamenidine, urapidil.

Examples of antihypotensives are: dihydroergotamine, dobutamine, dopamine, etilefrine, norepinephrine, norfenefrine.

Examples of anticoagulants are: acenocoumarol, dalteparin sodium, enoxaparin, heparin, heparinoids, hirudin, lepirudin, nadroparin, parnaparin, phenprocoumon, reviparin, tinzaparin, warfarin.

Examples of antimycotics are: amorolfin, amphotericin B, bifonazole, chlormidazole, ciclopiroxolamine, clotrimazole, croconazole, econazole, fenticonazole, fluconazole, griseofulvin, isoconazole, itraconazole, ketoconazole, miconazole, naftifine, nystatin, omoconazole, oxiconazole, terbinafine, terconazole, tioconazole, tolnaftate.

Examples of anti-rheumatics are: acemetacin, azapropazone, benorylate, bumadizone, carprofen, choline salicylate, diclofenac, diflunisal, etofenamate, felbinac, fenbufen, fenoprofen, flufenamic acid, flurbiprofen, ibuprofen, indometacin, isoxicam, ketoprofen, lonazolac, mefenamic acid, meloxicam, mofebutazone, nabumetone, naproxen, nifenazone, niflumic acid, oxyphenbutazone, phenylbutazone, piroxicam, pirprofen, proglumetacin, pyrazinobutazone, salsalate, sulindac, suxibuzone, tenoxicam, tiaprofenic acid, tolmetin, auranofin, aurothioglucose, aurothiomalate, aurothiopolypeptide, chloroquine, hydroxychloroquine, penicillamine, ademetionine, benzydamine, bufexamac, famprofazone, glucosamine, oxaceprol.

Examples of antitussives are: benproperine, butamirate, butetamate, clobutinol, clofedanol, codeine, dextromethorphan, dihydrocodeine, hydrocodone, isoaminil, sodium dibunate, noscapine, oxeladine, pentoxyverine, pholcodine, pipazetate.

Examples of appetite suppressants are: amfepramone, fenfluramine, fenproporex, levopropylhexedrine, mazindole, mefenorex, metamfepramone, norephedrine, norpseudoephedrine.

Examples of beta-receptor blockers are: acebutolol, alprenolol, atenolol, betaxolol, bisoprolol, bopindolol, bupranolol, carvedilol, celiprolol, labetalol, levobunolol, mepindolol, metipranolol, metoprolol, nadolol, oxprenolol, penbutolol, pindolol, propranolol, sotalol.

Examples of bronchospasmolytics/anti-asthmatics are: bambuterol, carbuterol, clenbuterol, epinephrine, fenoterol, hexoprenaline, ipratropium bromide, isoetarine, orciprenaline, oxitropium bromide, pirbuterol, procaterol, reproterol, salbutamol, salmeterol, terbutaline, theophylline, tolbuterol.

Examples of calcium antagonists are: amlodipine, felodipine, isradipine, nicardipine, nifedipine, nilvadipine, nitrendipine, nisoldipine, verapamil.

Examples of cholagogues are: anethole trithione, azintamide, chenodeoxycholic acid, dehydrocholic acid, hymecromone, piprozoline, ursodeoxycholic acid.

Examples of cholinergics/cholinolytics are: aceclidine, acetylcholine, carbachol, cyclopentolate, distigmine, edrophonium, emepronium, homatropine, methantheline, neostigmine, pilocarpine, propantheline, propiverine, pyridostigmine, tiropicamide.

Examples of diuretics are: acetazolamide, amiloride, bendroflumethiazide, bumetanide, chlorothiazide, chlorthalidone, clopamide, etaacrynic acid, furosemide, hydrochlorothiazide, triamterene, xipamide.

Examples of circulation-promoting agents/nootropics are: buflomedil, buphenine, dextran 40, dihydroergotoxin, iloprost, meclofenoxate, nicergoline, nicotinic acid, pentifylline, piracetam, piribedil, pyritinol, tolazoline, viquidil.

Examples of enzymes/inhibitors/transport proteins are: antithrombin III, aprotinine, carnitine, clavulanic acid, dornase alfa, sulbactam.

Examples of expectorants are: acetylcysteine, ambroxol, bromhexine, carbocysteine, colfosceril, surfactant (from bovine liver), surfactant (from pigs'lung).

Examples of antipodagrics are: allopurinol, benzbromarone, colchicine, probenecid, sulfinpyrazone.

Examples of glucocorticoids are: betamethasone, budesonide, cloprednol, cortisone, dexamethasone, flunisolide, fluticasone, hydrocortisone, methylprednisolone, paramethasone, prednisolone, prednisone, prednylidene, triamcinolone.

Examples of hemostyptics are: adrealon, blood clotting factor VII, blood clotting factor VIII, blood clotting factor IX, blood clotting factor XIII, carbazochrome, etamsylate, fibrinogen, collagen, menadiol, menadione, protamine, somatostain, thrombin, thromboplastin.

Examples of pituitary/hypothalamus hormones and inhibitors are: argipressin, choriogonadotropin, desmopressin, felypressin, gonadorelin, lypressin, menotropin, ornipressin, quinagolide, terlipressin, thyrotrophin.

Examples of immunotherapeutics and cytokines are: aldesleukin, azathioprine, BCG, ciclosporin, filgrastim, interferon alfa, interferon beta, interleukin-2, muromonab-CD3, tacrolismus, thymopentin, thymostimulin.

Examples of cardioactives are: acetyldigitoxin, acetylödiagoxin, convallatoxin, digitoxin, digoxin, gitoformate, lanatoside, meproscillarine, metildigoxin, pengitoxin, peruvoside, proscillaridine, strophanthin, thevetine, amrinone, enoximone, milrinone.

Examples of coronary agents are: carbocromene, isosorbide dinitrate, nitroglycerin, pentaerythrityl tetranitrate.

Examples of laxatives are: bisacodyl, danthrone, docusate, glycerol, lactulose, magnesium sulfate, sodium picosulfate, sodium sulfate, paraffinum subliquidum, phenolphthalein, castor oil, sorbitol.

Examples of hepatotherapeutics are: choline, citiolone, myo-inositol, slymarin.

Examples of hypolipidemics are: acipimox, bezafibrate, clofibrate, etofibrate, fluvastatin, lovastatin, pravastatin, simvastatin.

Examples of local anesthetics are: articaine, benzocaine, bupivacaine, butanilicaine, chloroethane, cinchocaine, cocaine, etidocaine, fomocaine, lidocaine, mepivacaine, myrtecaine, oxethazaine, oxybuprocaine, polidocanol, prilocaine, procaine, proxymetacaine, quinisocaine, tetracaine.

Examples of gastrointestinal agents are: bismuth subcitrate, bromopride, carbenoxolone, cimetidine, domperidone, famotidine, metoclopramide, nizatidine, omeprazole, proglumide, ranitidine, roxatidine, sucralfate, sulfasalazine.

Examples of migraine agents are: ergotamine, lisuride, naratriptan, pizotifen, sumatriptan, zolmitriptan.

Examples of muscle relaxants are: alcuronium, atracurium, baclofen, carisoprodol, chlormezanone, Clostridium toxin, Botulinum toxin A,

Examples of parathyroid gland therapeutics/calcium metabolism regulators are: clodronic acid, dihydrotachysterol, Glandulae parathyreoideae, pamidronic acid.

Examples of neuroleptics are: benperidol, chlorpromazine, droperidol, flugheanzine, haloperiol, melperone, promethazine, zuclopenthiozol

Examples of anti-Parkinson agents are: amantadine, benserazide, benzatropine, biperidene, bornaprine, bromocriptine, cabergoline, carbidopa, dihydroergocriptine, levodopa, metixene, pergolide, pramipexol, ropinirol, tolcapone.

Examples of psychostimulants are: amfetaminil, deanol, fencamfamine, fenetylline, kavaine, methylphenidate, pemoline, prolintane.

Examples of thyroid gland therapeutics are: carbimazole, Glandulae thyreoideae, iodine, iodide, levothyroxine, liothyronine, methylthiouracil, perchlorate, prolonium iodide, propylthiouracil, radio-iodine, thiamazole.

Examples of sedatives/hypnotics are: amobarbital, chloral hydrate, clomethiazole, glutethimide, hexobarbital, methaqualone, methyprylone, pentobarbital, scopolamine, secbutabarbital, secobarbital, vinylbital, zolpidem, zopiclone.

Examples of sex hormones are: chlorotrianisene, clomifene, clostebol, cyproterone, drostanolone, epimestrol, estradiol, estriol, estrone, ethinylestradiol, flutamide, fosfestrol, conjugated estrogens, medroxyprogesterone, mesterolone, mestranol, metenolone, methyltestosterone, nandrolone, oxymetholone, polyestradiol phosphate, quinestrol, stanozolol, testosterone.

Examples of spasmolytics are: atropine, butylscopolamine, flavoxate, glycopyrronium, mebeverine, methylscopolamine, oxybutynine, tiropramide, trospium.

Examples of platelet aggregation inhibitors are: abciximab, acetylsalicylic acid, dipyridamole, ticlopidine.

Examples of tranquillizers are: alprazolam, bromazepam, brotizolam, buspirone, camazepam, chlordiazepoxide, clobazam, clonazepam, clorazepate, clotiazepam, diazepam, flunitrazepam, flurazepam, hydroxyzine, ketazolam, loprazolam, lorazepam, lormetazepam, medazepam, meprobamate, metaclazepam, midazolam, nitrazepam, oxazepam, oxazolam, prazepam, temazepam, tetrazepam, triazolam.

Examples of urologicals are: finasteride.

Examples of Varia are: dapiprazole, diethyltoluamide, lipoic acid.

Examples of venologicals are: aescin, calcium dobesilate, coumarin, diosmin, rutoside, troxerutin.

Examples of virustatics are: aciclovir, cidofovir, didanosin, famciclovir, foscarnet, ganciclovir, lamivudine, ritonavir, zalcitabine, zidovudine.

Examples of vitamins are: alfacalcidol, allithiamine, ascorbic acid, biotin, calcifediol, calcitriol, cholecalciferol, cyanocobalamin, ergocalciferol, folic acid, hydroxocobalamin, nicotinamide, pantothenic acid, phytomenadione, pyridoxine, retinol, riboflavine, thiamine, tocopherol, transcalcifediol.

Examples of cytostatics are: aclarubicin, altretamine, aminoglutethimide, amsacrine, asparaginase, bleomycin, busereline, busulifan, carboplatin,carmustin, chlorambucil, cladribine, cisplatin, cyclophosphamide, cytarabine, dacarbazine, daunorubicin, diethylstilbestrol, docetaxel, doxorubicin, epirubicin, etoposide, fludarabin, fluorouracil, gemcitabin, goserelin, hydroxycarbamide, idarubicin, ifosfamide, lomustin, melphalan, mercaptopurine, mesna, methotrexate, miltefosin, mitomycin, mitoxantrone, panorex, paclitaxel, plicamycin, tamoxifen, tegafur, thiotepa, tioguanine, topotecan, triptoreline, vinblastine, vincristine, vindesine, zorubicin.

The composition according to the invention can also contain further excipients. “Excipients” is understood as meaning, for example, the following substance which are non-limiting for the present invention, however: water-insoluble excipients or mixtures thereof, such as lipids, inter alia fatty alcohols, e.g. cetyl alcohol, stearyl alcohol and cetostearyl alcohol; glycerides, e.g. glyceryl monostearate or mixtures of mono-, di- and triglycerides of vegetable oils; hydrogenated oils, such as hydrogenated castor oil or hydrogenated cottonseed oil; waxes, e.g. beeswax or carnauba wax; solid hydrocarbons, e.g. paraffin or mineral wax; fatty acids, e.g. stearic acid; certain cellulose derivatives, e.g. ethylcellulose or acetylcellulose; polymers or copolymers, such as polyalkylenes, e.g. polyethylene, polyvinyl compounds, e.g. poly(vinyl chloride) or poly(vinyl acetate), and also vinyl chloride-vinyl acetate copolymers and copolymers with crotonic acid, or polymers and copolymers of acrylates and methacrylates, e.g. copolymers of acrylic acid esters and methyl methacrylate; or surfactants, such as, for example, polysorbate 80 or docusate.

Aside from said excipients and active compounds, the composition according to the invention can additionally comprise fillers, disintegrants, binders and lubricants and also vehicles which do not have any decisive influence on the release of active compound. Examples are, inter alia, bentonite (aluminum oxide-silicon oxide hydrate), silicic acid, cellulose (customarily microcrystalline cellulose) or cellulose derivatives, e.g. methylcellulose, sodium carboxymethylcellulose, sugars, such as lactose, starches, e.g. cornstarch or derivatives thereof, e.g. sodium carboxy-methylstarch, starch leister, phosphoric acid salts, e.g. di- or tricalcium phosphate, gelatine, stearic acid or suitable salts thereof, e.g. magnesium stearate or calcium stearate, talc, colloidal silica and similar excipients.

The composition according to the invention comprises the compounds described preferably in a pulverulent embodiment. i.e., the composition can be present as an adsorbate, beadlet powder, granules, pellet, extrudate and/or combinations thereof. Likewise, use forms in which the particles are coated are conceivable.

The compositions according to the invention, which are preferably present in powder form, can be prepared using methods known per se. This includes, for example, the preparation of spray formulations. A process which can be employed and plant for this is described, for example, in EP 0 074 050 B1.

In addition to this manner of preparation, further process variants are also conceivable. These include, for example, spray drying processes or the preparation of adsorbates in fluidized beds.

For preparation of a pulverulent composition according to the invention, it is possible, for example, to prepare a solution in water of the polymers having a low degree of esterification and to thicken it, for example, with addition of calcium salts. By incorporating air and optionally after addition of surfactants, a gel or foam can be obtained. By means of freezing and subsequent freeze drying, a dry gel or dry foam (sponge) is prepared from the alginate gel or foam. The further compounds, which must be swellable according to the invention, can be prepared in an analogous manner.

Beside the addition of inorganic or organic calcium salts, for example, calcium chloride or calcium gluconate, the use of magnesium salts is also conceivable, and also of mixtures of various physiologically acceptable divalent or trivalent cations.

The foam described can be employed, however, without prior freezing or drying. In this connection, the foam is taken in the form of a spongy material, preferably in compressed form. In the stomach, the spongy material expands and causes a satiation effect. Foam-like structures of this type are disclosed, for example, in DE 4025912 and DE 19942417.

The preparation of granules can be achieved by introducing vehicles and/or spray-dried powder and also, if appropriate, additives into a mixer and producing compact granules by addition of the active components and/or binder and/or additives. Mixers preferably employed in this process are, for example, paddle mixers or plowshare mixers. The liquid components can, for example, be applied drop by drop or sprayed on such that a pasty, sticky phase results. By means of suitable choice of the speed of rotation of the mixer tools and/or fast-running knives, the pasty phase is dispersed and compact granules result. Very large lumps are disintegrated by the mixer tools and knives, and on the other hand, fine powders are agglomerated. By addition of enveloping layers can be carried out at a later stage in the mixer at a relatively low speed of rotation of the mixing apparatus and standing knives or in a constructionally related mixer connected at a later stage.

The composition according to the invention can be prepared in various customary administration forms. For instance, it can be present, for example, in the form of tablets, capsules, coated tablets, as granules or powders or other embodiments.

The composition according to the invention can be used for producing a satiation effect, for weight reduction and for the regulation of cholesterol metabolism.

Moreover, it is suitable for the preparation of a composition for producing a satiation effect, or weight reduction and for regulation of cholesterol metabolism.

Furthermore, the composition described is suitable for the delayed release of nutrients or active compounds or mixtures thereof. Accordingly, compositions for the delayed release of nutrients or active compounds or mixtures thereof can be prepared from the compositions described.

The composition according to the invention makes possible improved nutrient and/or active compound uptake with simultaneous achievement of a weight reduction. Satiation effect. The swellable compounds, which preferably display their action in the form of a gel, caused a filling of the stomach, such that a satiation effect occurs. The compounds (nutrients and/or active compounds) enclosed in the swollen gel article are released gradually, i.e. in delayed form. On account of this, food can be continuously supplied to the body from the swollen gel article filling the stomach. i.e., as long as a feeling of satiation is present due to the gel article, the body nevertheless receives food. In order to prevent disease symptoms or to treat such symptoms, according to the invention when using active compounds their delayed release is moreover possible simultaneously. According to the invention, the composition, however, is also suitable for exclusive medicinal use, i.e. for any form of delayed release compositions.

In one variant of the invention, consumption takes place after the composition has been prepared in liquid. By stirring in the composition, pre-swelling takes place, which continues in the stomach after consumption. Depending on the degree of pre-swelling, the stirred-in composition can be drunk or consumed by means of a spoon.

Pre-swelling preferably takes place by stirring 1-20 g., preferably 2-10 g., particularly preferably 3-6 g of the swellable compound into 100 to 500 ml, preferably 200 to 400 ml, particularly preferably 250 to 350 ml of liquid.

The present invention is characterized in greater detail by means of the figures, which, however, do not have a limiting effect on the invention.

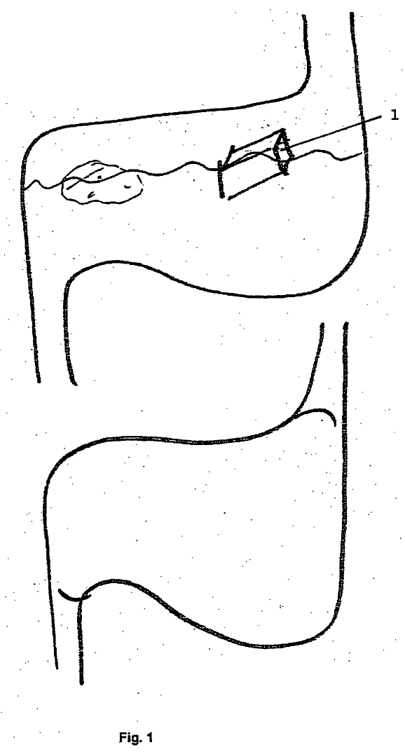

FIG. 1 shows the use of delayed-release compositions known from the prior art. It is clear to see that the articles (1) float in the swollen state. I.e., the previously known compositions may not be able to fill the stomach.

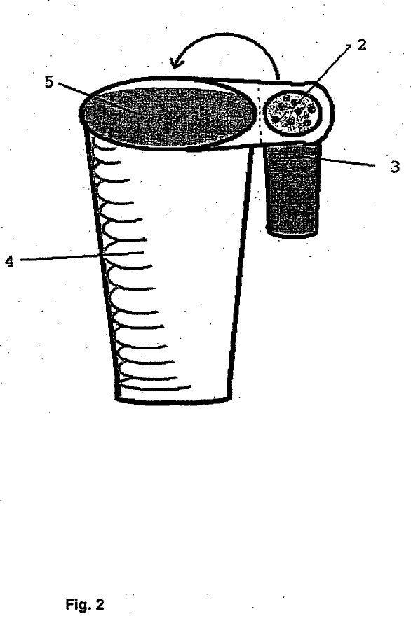

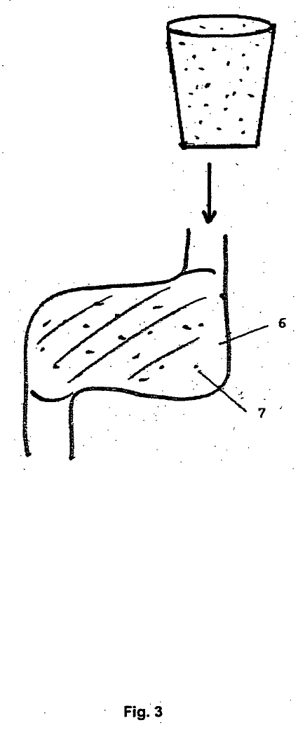

In FIGS. 2 and 3, the use of the composition according to the invention (2) is shown. The composition according to the invention is stirred in a vessel.

In a preferred embodiment, a beaker from FIG. 2 is used, which consists of two containers (3) and (4). A liquid (5), such as, for example, water, fruit juice, milk, coffee or tea beverages, is situated in or filled into one container (4), while the other container (3) contains the swellable composition according to the invention (2). Both containers are connected to one another. The opening of the container containing the composition (2) can be brought over the opening of the container containing the liquid. The composition according to the invention is thus introduced into the liquid.

Depending on the nature and amount of the composition employed, a gelatinous or liquid mass is formed. A beaker containing this mass is shown schematically in FIG. 3. After this mass is consumed, a body of gel (6) is formed in the stomach, which completely fills this. The substances (7) enclosed in the body of gel are gradually released in the direction of the pylorus.

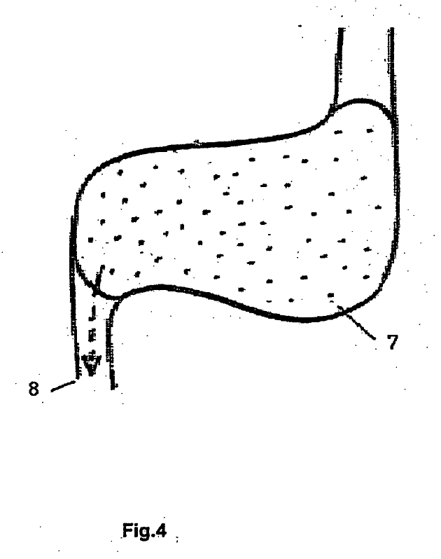

This process of the release of the active compounds 3 in the direction of the pylorus (8) is shown in FIG. 4.

1. A composition for producing a satiation effect and for weight reduction comprising at least one compound which is swellable and comprises nutrients or active compounds or mixtures of nutrients and active compounds.

2. The composition as claimed in claim 1, wherein the swellable compound comprises anionic polymers.

3. The composition as claimed in claim 1, wherein the anionic polymers present are polysaccharides.

4. The composition as claimed in claim 1, wherein it comprises polyuronic acid-containing polysaccharides.

5. The composition as claimed in claim 1, wherein the polyuronic acid-containing polymers are alginic acid or its derivatives or cellulose or its derivatives or mixtures of said compounds.

6. The composition as claimed in claim 1, wherein the polymers having a low degree of esterification present are pectins, xanthan, tragacanth, chondroitin sulfate or mixtures of these compounds.

7. The composition as claimed in claim 1, wherein the polymers having a low degree of esterification present are pectins, xanthan, tragacanth, chondroitin sulfate or mixtures of these compounds.

8. The composition as claimed in claim 1, wherein the nutrients present are vitamins, amino acids, minerals or trace elements.

9. The composition as claimed in claim 1, wherein the active compounds present are vitamins, trace elements or pharmaceutical active compounds.

10. The composition as claimed in claim 1, wherein it is present in the form of a powder.

11. The use of a composition as claimed in claim 1 for producing a satiation effect and for weight reduction.

12. The use of a composition as claimed in claim 1 for the delayed release of nutrients or active compounds or mixtures thereof.

13. The use of a composition as claimed in claim 1 for the preparation of a composition for producing a satiation effect and for weight reduction.

14. The use of a composition as claimed in claim 1 for the preparation of a composition for the delayed release of nutrients or active compounds or mixtures thereof.

15. The use as claimed in claim 14, wherein a composition as claimed in claim 1 is stirred into a liquid, preferably water, fruit juice, milk, coffee or tea beverages.

16. The use as claimed in claim 15, wherein a beaker consisting of two containers (3) and (4) is employed, where a liquid (5) is situated in or filled into one container (4), and the other container (3) contains the composition (2) as claimed in claim 1, which composition is introduced into the liquid (5) by bringing the opening of the container containing the composition (2) over the opening of the container containing the liquid.
